(19) United States
(12) Patent Application Publication
(10) Pub. No.: US 2006/0225307 A1
(43) Pub. Date: Oct. 12, 2006

(54) Colored spa shoes

(76) Inventor: Sung-Po Huang, Yonghe City (TW)

Correspondence Address: Sung-Po HUANG, P.O. Box No. 6-57, Junghe, Taipei, 235, TW

(21) Appl. No.: 11/100,587

(22) Filed: Apr. 7, 2005

(51) Int. Cl. A61F 5/14

(52) U.S. Cl. 36/141; 36/153

(57) ABSTRACT

Color SPA shoes each include an insole of a size compatible to a vamp thereof, and is glued or sewed onto the vamp. A fluid infused into the insole is circulated therein when walking to provide a foot with massaging effects. Each step creates expansion and contraction of a space between the foot and the insole to ventilate and accelerate exudation of perspiration. Colloids in the insoles are composed of light transmissive material and enable different colors of fluids to match with different colors and styles of shoes, thereby achieving a stylish appearance.

### BACKGROUND OF THE INVENTION

(a) Field of the Invention

The invention relates to colored spa shoes, and more particularly pertains to colored spa shoes having insoles, which are in a size compatible to vamps of the spa shoes and are glued or sewed onto the vamps.

(b) Description of the Prior Art

A vamp A1 of shoe A of prior art typically is a hard surface, which causes discomforts after long-distance walking. Although the improved vamp A1 made with a soft material is targeted to fit the sole of the foot better, it is still rather ineffective in relieving fatigue from walking. Nowadays, parts of the vamp may use granular structures for providing massaging effects. However, the granular structures are fixed in specific spot and yet cause pain to the foot after long-distance walking.

Therefore, it is a vital task of the invention as how to provide spa shoes having satisfactory massaging effects.

### SUMMARY OF THE INVENTION

The primary object of the invention is to provide colored SPA shoes having massaging effects and capable of accelerating exudation of perspiration in each step. To accomplish the aforesaid object, the invention comprises an insole in a size compatible to a size of the vamp that is glued or sewed onto a shoe body. When walking, a fluid inside of the insole provides massaging effects to the foot. Meanwhile, each step creates expansion and contraction of spaces between the foot and the insole; thereby discharging air and accelerating exudation of perspiration.

To better understand characteristics, effects and functions of the invention, detailed descriptions of a preferred embodiment shall be given with the accompanying drawings below.

### BRIEF DESCRIPTION OF THE DRAWINGS

FIG. 1 shows an elevational view according to the prior art.

FIG. 2 shows an elevational view according to the invention.

FIG. 3 shows exploded elevational view according to the invention.

FIG. 4 shows a sectional view of the insole according to the invention.

FIG. 4a shows an enlarged view of FIG. 4 according to the invention.

FIG. 5 shows a schematic view illustrating motions of an embodiment according to the invention.

FIG. 6 shows another schematic view illustrating motions of an embodiment according to the invention.

FIG. 7 shows a schematic view of another embodiment according to the invention.

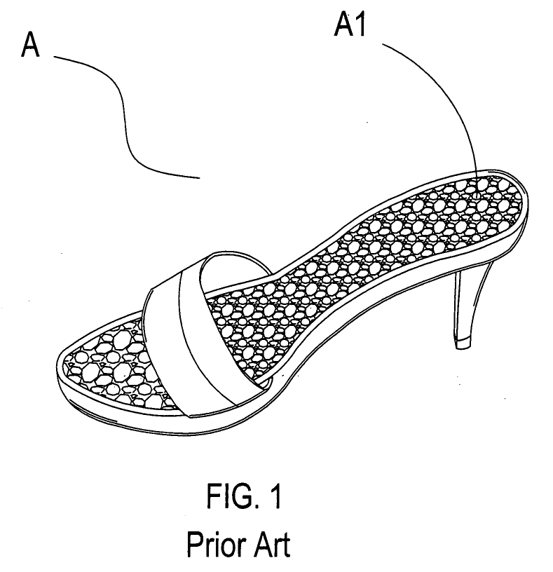

### DETAILED DESCRIPTION OF THE PREFERRED EMBODIMENTS

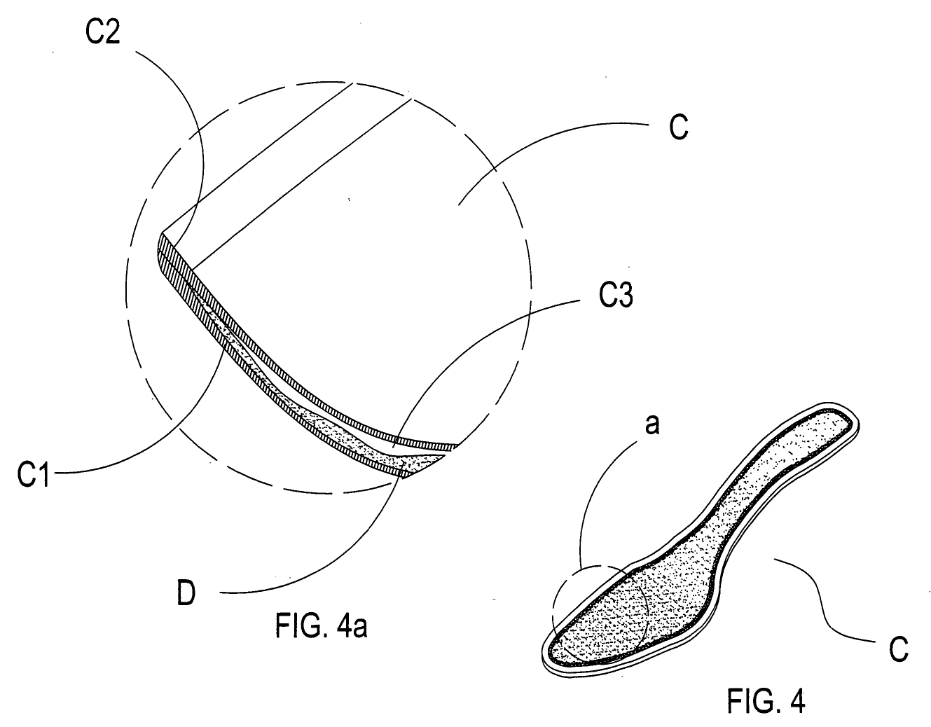

According to FIG. 4 and FIG. 4a, an insole C is consisted of a plurality of colloids C1 bonds together therein, such that a side edge C2 to allowed with coherence. A fluid D is infused into a saccate substance C3 while leaving a buffering space within which the fluid D can circulate.

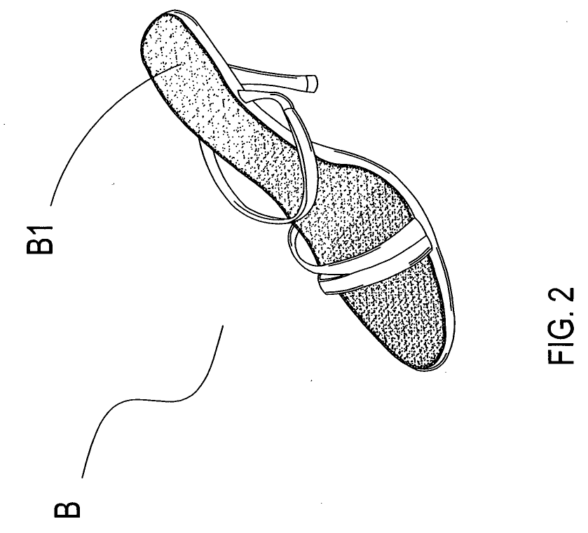

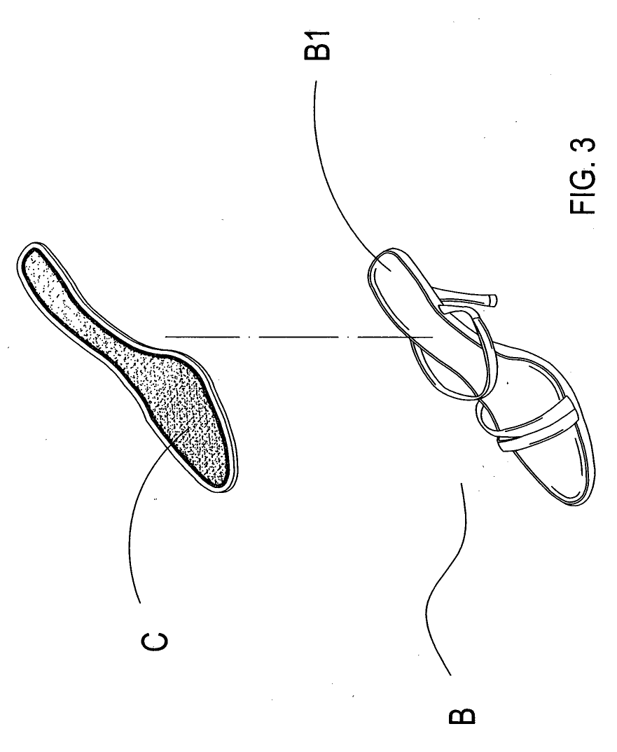

Further, the insole C, in a size compatible to the vamp B1, is sewed onto a shoe body B through the side edge C2 or glued to a vamp B1 as shown in FIG. 2 and FIG. 3. The fluid D is infused into the insole C therein circulates with human walking motion to provide massaging effects to a foot. Meanwhile, each step creates expansion and contraction of spaces between the foot and the insole C; thereby discharging air and accelerating exudation of perspiration.

The colloids C1 are composed of a light-transmissive material. Different colors of the fluid D can be infused to match different colors or styles of shoe body B to achieve a stylish appearance.

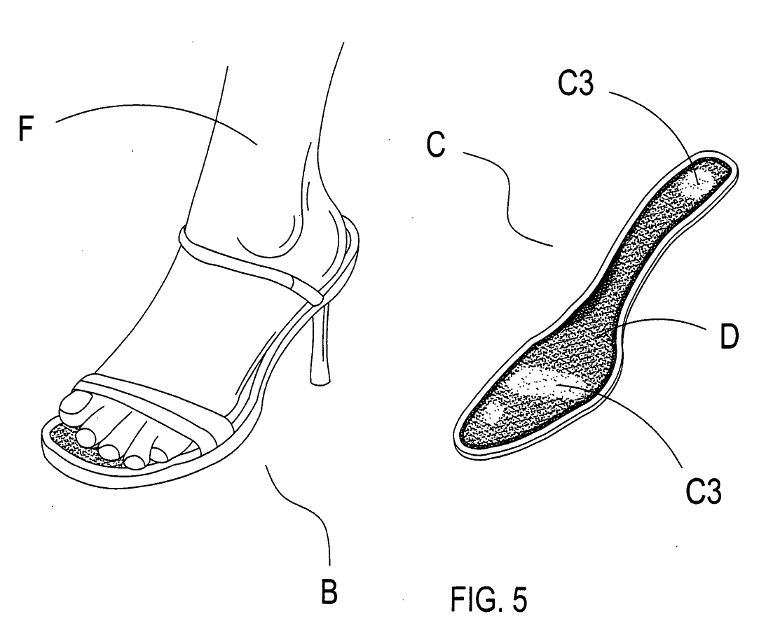

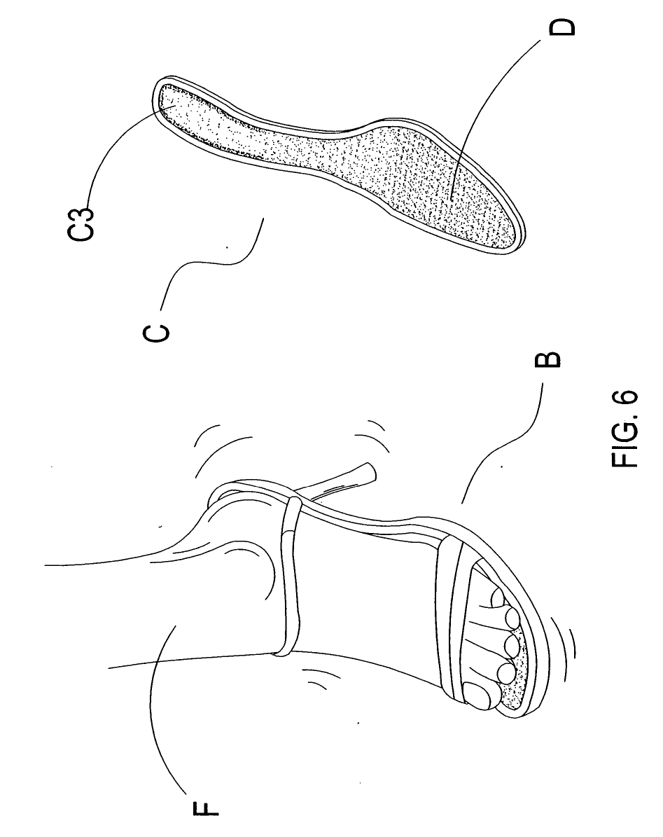

As shown in FIG. 5 and FIG. 6, a foot F is wearing a shoe B and an insole C is in contact with the foot F. When standing still, a fluid D spreads in the insole C evenly. When walking, the shoe B is in motion along with the foot F, which causes the fluid D to circulate in the insole C. While the fluid D circulating at a surface of the insole C undulates and massages, fatigue from long-distance walking of the foot F is thus relieved. A contact surface of the foot F and the insole D can ventilate and accelerate exuding perspiration of the foot F to reduce odors and avoid germs.

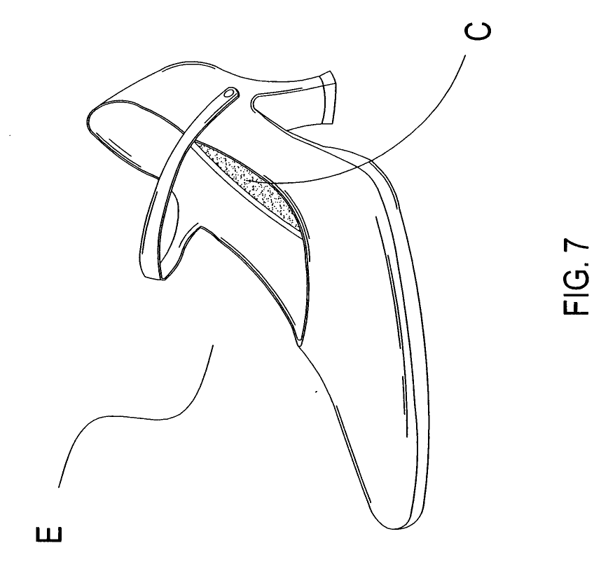

Referring to FIG. 7, a shoe E comprises an insole C, and a color of the insole C may vary to match that of the shoe E. Furthermore, the insole C is applicable to sneakers, golf shoes, sandals, hiking shoes, leather boots and all other types of shoes.

To emphasize novelty and practicability of the invention, the prior art and the invention are analyzed as below:

Disadvantages of the Prior Art:

1. Hard vamp causes discomforts to foot when walking.

2. Soft vamp does not relieve fatigue of the foot when walking.

3. Vamp with granular structure causes pain to foot after long-distance walking.

Advantages of the Invention:

1. Fluid achieves massaging effects to the foot when walking.

2. Fluid circulation avoids long-term on-spot pressure to foot when walking.

3. The spa shoes conform to ergonomics of human walking motion.

4. Various choices of colors of the fluid are provided to match with shoe body for stylish appearance.

5. Circulation of fluid enables the compression of vapor between the foot and insole to achieve ventilation.

6. The spa shoes enhance comfort when walking.

7. The spa shoes offers novelty and practicability.

8. The spa shoes increase industrial competitiveness.

It is of course to be understood that the embodiment described herein is merely illustrative of the principles of the invention and that a wide variety of modifications thereto may be effected by persons skilled in the art without departing from the spirit and scope of the invention as set forth in the following claims.

What is claimed is:

1. Color spa shoes each comprising:
an insole that is light transmissive, wherein a saccate substance is infused with a colored fluid and has a side-edge thereof being cohered; and
a shoe provided a vamp having the insole thereon, and a fluid placed inside the insole and circulated with the motion of the shoe.

2. The color spa shoes in accordance with claim 1, wherein each shoe and the insole are sewed, glued, joined or assembled with all related substances using similar techniques.

3. The color spa shoes in accordance with claim 1, wherein the shoe is applied to sneakers, leather shoes, high-heeled shoes, casual walking shoes, golf shoes, sandals, hiking shoes, leather boots and all other types of shoes.
